(19) United States
(12) Patent Application Publication
(10) Pub. No.: US 2025/0381048 A1
(43) Pub. Date: Dec. 18, 2025

(54) COMPOSITE STENT WITH VARIABLE DIAMETER AND CROSS SECTION

(71) Applicants: BOARD OF TRUSTEES OF THE UNIVERSITY OF ARKANSAS, Little Rock, AR (US); BIOVENTURES, LLC, Little Rock, AR (US)

(72) Inventors: Morten Jensen, Fayetteville, AR (US); Kaitlyn Elmer, Fayetteville, AR (US); Barry Uretsky, Fort Smith, AR (US); Adib Chaus, Des Plaines, IL (US)

(73) Assignees: BOARD OF TRUSTEES OF THE UNIVERSITY OF ARKANSAS, Little Rock, AR (US); BIOVENTURES, LLC, Little Rock, AR (US)

(21) Appl. No.: 19/236,451

(22) Filed: Jun. 12, 2025

Related U.S. Application Data
(60) Provisional application No. 63/659,261, filed on Jun. 12, 2024.

(51) Int. Cl. A61F 2/89; A61F 2/06; A61F 2/954; A61F 2/958

(52) U.S. Cl. CPC A61F 2/89; A61F 2/954; A61F 2/958; A61F 2002/065; A61F 2250/0039

(57) ABSTRACT

The present disclosure pertains to a stent that includes a proximal end and a distal end; and a plurality of interconnected and expandable rings positioned adjacent to one another from the proximal end to the distal end. Each ring is connected to and spaced apart from at least one adjacent ring through one or more connecting units. Additionally, the interconnected rings form a cavity. The rings at or near the proximal end include different diameters than the rings at or near the distal end. The present disclosure also pertains to methods of implanting a stent of the present disclosure in a blood vessel of a subject through the following steps: (1) positioning the stent at a desired location within the blood vessel; and (2) expanding the stent from a contracted configuration to an expanded configuration to secure the stent to the blood vessel wall.

### CROSS-REFERENCE TO RELATED APPLICATIONS

This application claims priority to U.S. Provisional Patent Application No. 63/659,261, filed on Jun. 12, 2024. The entirety of the aforementioned application is incorporated herein by reference.

### BACKGROUND

A need exists for improved stents for effective implantation at or near vessel bifurcations. Numerous embodiments of the present disclosure aim to address this need.

### SUMMARY

In some embodiments, the present disclosure pertains to a stent. In some embodiments, the stent includes a proximal end and a distal end, and a plurality of interconnected and expandable rings positioned adjacent to one another from the proximal end to the distal end. In some embodiments, each ring is connected to and spaced apart from at least one adjacent ring through one or more connecting units. In some embodiments, the interconnected rings form a cavity. In some embodiments, the rings at or near the proximal end include different diameters than the rings at or near the distal end.

Additional embodiments of the present disclosure pertain to methods of implanting a stent of the present disclosure in a blood vessel of a subject. In some embodiments, such methods include (1) positioning the stent at a desired location within the blood vessel; and (2) expanding the stent from a contracted configuration to an expanded configuration to secure the stent to the blood vessel wall.

### DRAWINGS

FIGS. 1A-1C illustrate a stent in accordance with various embodiments of the present disclosure.

FIG. 2 depicts a proximal-to-distal stenting strategy for a tapered bifurcation stent of the present disclosure. The stent covers the proximal vessel of the bifurcation up to the bifurcation carina. The stent is tapered to cover the widening that occurs at the side-branch. Additional stents can be placed distally as needed, with minimal stent overlap and full coverage of the vessel. The stent is deployed over a dual-wire balloon.

FIGS. 3A-3D provide images of a stent of the present disclosure. FIG. 3A provides an image of a stent crimped onto a balloon. FIG. 3B provides an image of a stent expanded “conically.” FIG. 3C provides an image of a stent expanded elliptically, with two “kissing” balloons. A long axis of the ellipse is shown. FIG. 3D provides an image of a stent expanded elliptically, with two “kissing” balloons. A short axis of the ellipse is shown.

FIG. 4 provides an image of a formed wire of the present disclosure attached to a pre-wielding layout. The “top” layers have 9 and 10 zig-zags, while the bottom layers have 8. The additional zig-zags allow for more expansion.

FIG. 5 provides an illustration of a wire sheet following laser-welding of connectors. The wire sheet has been wrapped around a mandrel and trimmed, before the seam was laser-welded to complete the stent.

FIG. 6 provides an image of a completed stent after expansion.

FIGS. 7A-7D2 provide images of stent expansion tests described in Example 2. FIG. 7A provides an image of a stent crimped onto a balloon used in Test 1. FIG. 7B provides an image of a stent and balloon inflated during Test 1. FIGS. 7C1 and 7C2 provide images of sample DKBI, including the long axis of the DKBI (FIG. 7C1) and the short axis (FIG. 7C2). FIGS. 7D1 and 7D2 provide examples of stents following inflation tests. FIG. 7D1 provides an example of a long axis of a stent while FIG. 7D2 provides an example of a short axis of a stent.

FIGS. 8A-8D provide results of balloon delivery tests described in Example 2. FIG. 8A shows an example of phantom used in the test. FIG. 8B shows an example of a balloon-catheter used in the test. FIG. 8C shows a stent and balloon within an artery. FIG. 8D shows a placed stent in an artery while the balloon is retracted.

FIGS. 9A-9C provide cross sections of bifurcation tests 1-3 described in Example 2. The bright white spots indicate stent struts. Test 1 and 3 saw stent edges nearest to the carina of the bifurcation.

FIG. 10 provides a MicroCT scan of a bifurcation test described in Example 2. The cross sections of various locations in the pMV are shown, including the distal stent edge and the proximal carina edge. The bright white spots are the prototype stent struts, the light grey layer surrounding the vessel void is the 3D printed material and the darker gray surrounding the vessel is the gel.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Methods and devices for treating and preventing arterial diseases have numerous limitations. For instance, a major treatment for obstructive coronary artery disease (CAD) is catheter-based revascularization known as percutaneous coronary intervention (PCI). Part of the procedure typically utilizes a balloon attached to a catheter. The balloon is inflated to enlarge the vessel lumen prior to implanting a drug-eluting stent, which has been shown to decrease the risk of re-blockage at the original stenosis site.

In fact, existing stents have limits to individual cell expansion. Stent overexpansion past these limits distorts stent architecture. Under-expansion, on the other hand, is associated with adverse short and long-term clinical outcomes.

Moreover, current cylindrical stents cannot easily accommodate bifurcated vessel areas without placing more than one metal layer within a bifurcated area. For instance, commercially available PCI balloons and stents are cylindrical in shape. However, at vessel bifurcations, balloons and stents do not match the local geometry.

Many approaches have been devised to implant stents at vessel bifurcations. However, none have proven to be completely satisfactory. Issues such as overlapping stents or stents not well-opposed to the vessel wall are a common deficiency that promote regrowth of tissue with consequent vessel obstruction. At the vessel bifurcation, the vessels expand outwards due to the main vessel splitting into two branches.

As such, a need exists for improved stents for effective implantation at or near vessel bifurcations. Numerous embodiments of the present disclosure aim to address this need.

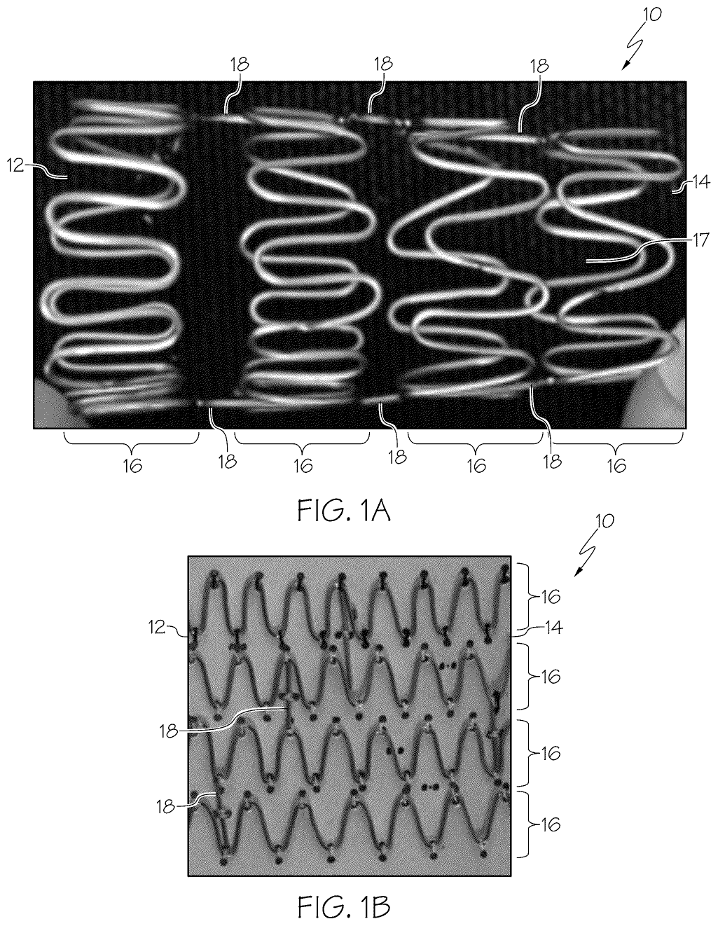

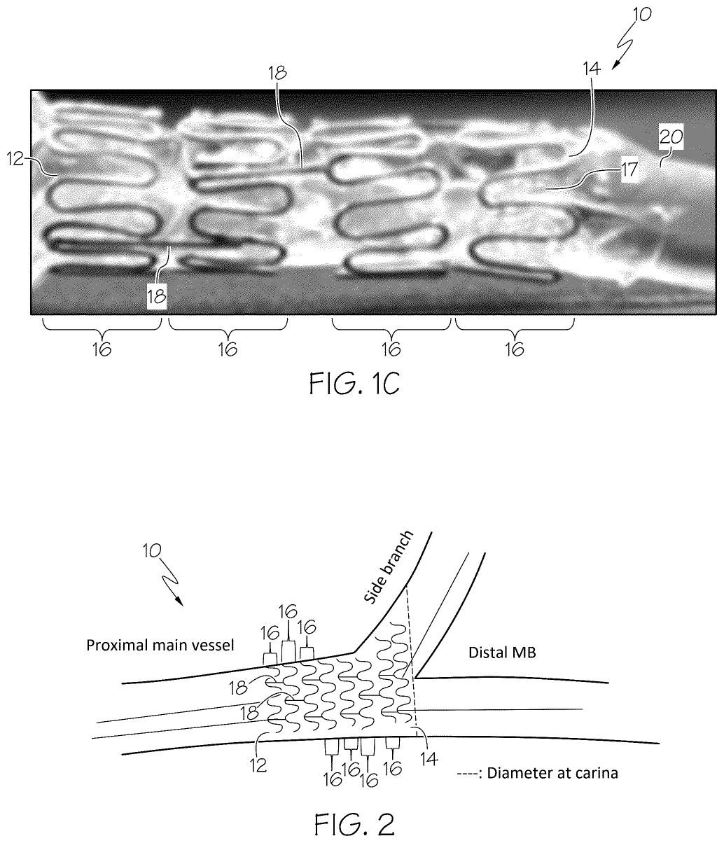

In some embodiments, the present disclosure pertains to a stent. With reference to FIGS. 1A-1C and 2 for illustrative purposes, the stents of the present disclosure may be illustrated as stent 10. In some embodiments, stent 10 includes a proximal end 12 and a distal end 14; and a plurality of interconnected and expandable rings 16 positioned adjacent to one another from the proximal end 12 to the distal end 14. In some embodiments, each ring 16 is connected to and spaced apart from at least one adjacent ring 16 through one or more connecting units 18. In some embodiments, the interconnected rings 16 form a cavity 17.

Additional embodiments of the present disclosure pertain to methods of implanting a stent of the present disclosure in a blood vessel of a subject. In some embodiments, such methods include (1) positioning the stent at a desired location within the blood vessel; and (2) expanding the stent from a contracted configuration to an expanded configuration to secure the stent to the blood vessel wall.

As set forth in more detail herein, the stents and methods of the present disclosure can have numerous embodiments. In particular, the stents of the present disclosure can have numerous structures and arrangements. Additionally, the methods of the present disclosure may be utilized to implant the stents of the present disclosure into various blood vessels of various subjects for various applications.

### Expanding Structures

In some embodiments, the stents of the present disclosure are operable to receive at least one expanding structure 20 in their cavity 17 and be expanded by expanding structure 20. In some embodiments, the stents of the present disclosure are operable to receive at least two expanding structures 20 in their cavity 17 and be expanded by the two expanding structures 20.

In some embodiments, the stents of the present disclosure also include the expanding structure 20. In some embodiments, the stent expansion methods of the present disclosure include introducing at least one expanding structure 20 into cavity 17 and expanding the expanding structure 20.

The stents of present disclosure can include various expanding structures. Additionally, the methods of the present disclosure can utilize various expanding structures to expand the stents of the present disclosure. For instance, in some embodiments, the expanding structure includes a balloon. In some embodiments, the expanding structure is tapered from a proximal end to a distal end. In some embodiments, the expanding structure includes a larger diameter at the proximal end than at the distal end. In some embodiments, the expanding structure includes a larger diameter at the distal end than at the proximal end.

In some embodiments, the expanding structure includes a first balloon and a second balloon, In some embodiments, the first balloon is operable to extend into a main branch of a bifurcated vessel. In some embodiments, the second balloon is operable to extend into a side branch of a bifurcated vessel.

In some embodiments, the individual rings 16 are connected by connecting units 18 around the expanding structure 20. In some embodiments, the stent is only operable to be expanded by an expanding structure.

### Rings

The stents of the present disclosure can include various numbers of rings. For instance, in some embodiments, the stents of the present disclosure include at least four rings. In some embodiments, the stents of the present disclosure include at least six rings. In some embodiments, the stents of the present disclosure include at least eight rings.

The rings of the present disclosure can include various sizes. For instance, in some embodiments, rings at or near a proximal end of a stent include different diameters than the rings at or near a distal end of the stent. In some embodiments, rings at or near a proximal end of a stent include larger diameters than the rings at or near a distal end of the stent. In some embodiments, rings at or near a proximal end of a stent include smaller diameters than the rings at or near a distal end of the stent.

The rings of the present disclosure may be composed of various materials. For instance, in some embodiments, the rings of the present disclosure include, without limitation, alloys, cobalt, chromium, or combinations thereof.

The stents of the present disclosure can include various ring shapes. For instance, in some embodiments illustrated in FIGS. 1A-1C and 2, the rings 16 include a sinusoidal shape. In some embodiments, the sinusoidal shape includes alternating peaks and troughs along the ring length. In some embodiments, rings 16 at or near the proximal end 12 include more peaks and troughs than the rings 16 at or near the distal end 14. In some embodiments, rings 16 at or near the distal end 14 include more peaks and troughs than the rings 16 at or near the proximal end 12.

### Connecting Units

The stents of the present disclosure can include various numbers of connecting units. For instance, in some embodiments, the stent includes less than three connecting units between each adjacent ring. In some embodiments, the stent includes one to three connecting units between each adjacent ring. In some embodiments, the stent includes one or two connecting units between each adjacent ring. In some embodiments, the stent includes two connecting units between each adjacent ring.

In some embodiments, the connecting units are operational to allow the expansion of each individual ring to a shape independently of other individual rings. In particular, in some embodiments, the connecting units between rings are minimal to allow the individual rings to expand to different shapes or diameters without being inhibited by the expansion of other rings.

The stents of the present disclosure can include various types of connecting units. For instance, in some embodiments, the connecting units include struts.

### Stent Geometries

The stents of the present disclosure can include various geometries. For instance, in some embodiments, the rings of the stents of the present disclosure are connected to one another through connecting units to form a unitary structure without bifurcations. In some embodiments, the stents of the present disclosure include a tapered structure from a proximal end to a distal end when the rings are expanded.

In some embodiments, the stent cavity includes a non-uniform diameter from the proximal end to the distal end when the rings are expanded. In some embodiments, the cavity includes a larger diameter at the proximal end than at the distal end when the rings are expanded. In some embodiments, the cavity includes a larger diameter at the distal end than at the proximal end when the rings are expanded. In some embodiments, the diameter ratio of the cavity at the distal end relative to the cavity at the proximal end ranges from 1.2 to 2 when the rings are expanded. In some embodiments, the diameter ratio of the cavity at the distal end relative to the cavity at the proximal end ranges from 1.5 to 2 when the rings are expanded. In some embodiments, the diameter ratio of the cavity at the distal end relative to the cavity at the proximal end is approximately 1.3 when the rings are expanded.

In some embodiments, stent cavities include different shapes. For instance, in some embodiments, the stent cavity includes a circular shape at the proximal end and an elliptical shape at the distal end when the rings are expanded.

### Active Agents

In some embodiments, the stents of the present disclosure may also be associated with one or more active agents that are releasable into blood vessels. For instance, in some embodiments, the active agents include tissue-inhibitor drugs.

### Implantation into Blood Vessels

The stents of the present disclosure may be suitable for implantation into various blood vessels. As such, the methods of the present disclosure may be utilized to implant the stents of the present disclosure into various blood vessels of subjects.

For instance, in some embodiments illustrated in FIG. 2, stent 10 may be implanted at or near vessel bifurcations. In some embodiments, stent 10 may cover a proximal vessel of a bifurcated vessel up to a bifurcation carina. In some embodiments, stent 10 may cover a widening at a side-branch of a bifurcated vessel. In some embodiments, one or more rings 16 at the distal end 14 may expand to a circular or elliptical shape to accommodate a vessel bifurcation area up to a carina between a main branch and a side branch.

### Stent Delivery Systems

In some embodiments, the stents of the present disclosure may be associated with a stent delivery system. In some embodiments, the stent implantation methods of the present disclosure occur through the utilization of a stent delivery system. In some embodiments, the stent delivery system includes: (1) a catheter operable for carrying the stent; (2) a navigating system operable for directing the stent to the blood vessel; and (3) an expanding structure operable for expanding the stent at a targeted location in the blood vessel. In some embodiments, the positioning of the stent at a desired blood vessel location includes: (1) obtaining images of the blood vessel, (2) using an imaging device associated with the stent delivery system to observe real-time positioning, and (3) actuating an expansion mechanism to expand the expanding structure and secure the stent at the target site as visualized in the images. In some embodiments, the expansion mechanism includes a fluid delivery conduit in communication with the expanding structure for delivering a fluid to expand the expanding structure.

### Applications

The stents and stent implantation methods of the present disclosure can have numerous applications. For instance, in some embodiments, stents of the present disclosure may be suitable for use as a coronary stent. In some embodiments, the stents of the present disclosure may be suitable for use as an arterial bifurcation stent. In some embodiments, the stents of the present disclosure may be suitable for use for pulmonary bifurcation.

In some embodiments, the stent implantation methods of the present disclosure may be utilized to treat or prevent a coronary artery disease. In some embodiments, the stent implantation methods of the present disclosure may be utilized for pulmonary bifurcation.

### Subjects

The stent implantation methods of the present disclosure may be utilized to implant stents into the blood vessels of various subjects. For instance, in some embodiments, the subject is a human being. In some embodiments, the subject is vulnerable to or suffering from a coronary artery disease.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Design and Validation of Stents for Vessel Bifurcations

This Example describes the placement of an improved stent in the main vessel of a bifurcated vessel and far enough into the bifurcation core to span the widest diameter of the bifurcation. For this method, stents and balloon sizes may be chosen to accommodate this outward expansion.

In particular, as illustrated in FIG. 2, this Example describes stents for vessel (e.g., coronary) bifurcations, which expand into the “core” of the bifurcation and are tapered to accommodate the bifurcation shape. The stents are designed to be a scaffold for tissue-inhibitor drugs with or without polymer. Proximally (i.e., in the main vessel), the stent balloon matches the diameter of the proximal vessel with the stent itself designed to accommodate the main vessel diameter (FIG. 2). Distally, the stent can expand to a circular or elliptical shape to accommodate the bifurcation area to the carina between main branch and side branch (FIG. 2). For example, a 16-21 mm stent can expand to 16 mm diameter proximally and 21 mm diameter distally, or in an elliptical expansion, an ellipse with long/short axes of 16 and 26 mm. In other words, the expansion ratio between large/small end of the stent is 1.3 or 1.6 for circular or elliptical expansion respectively.

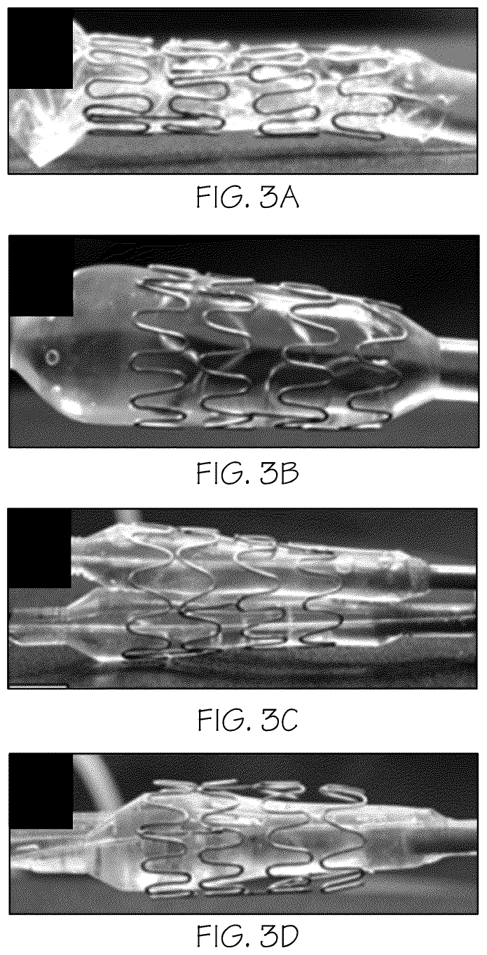

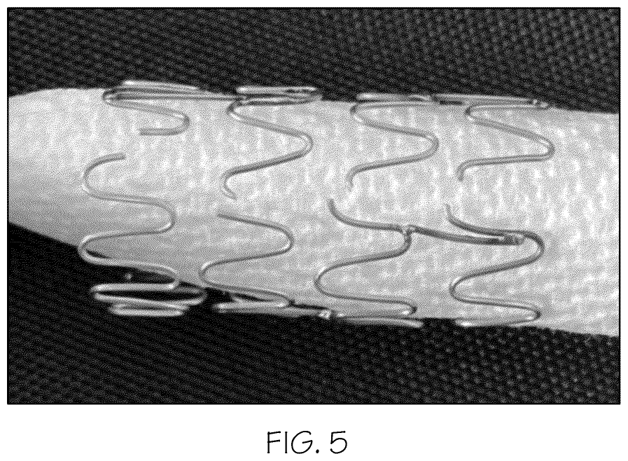

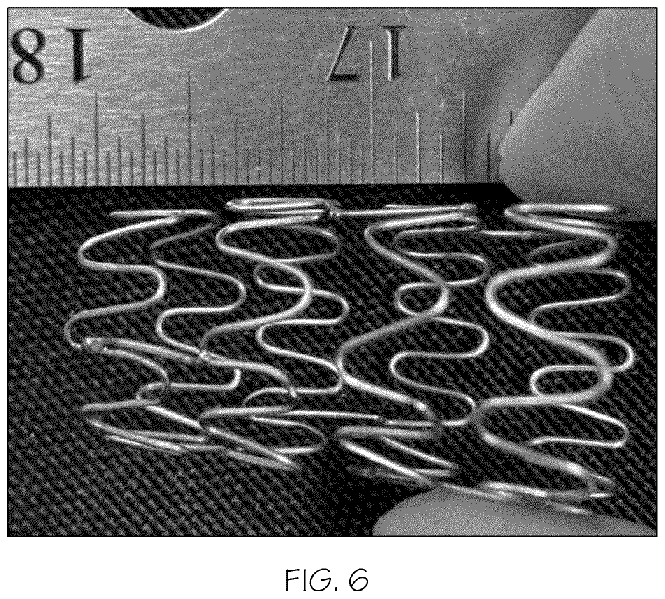

As illustrated in FIGS. 3A-3D, 4-6 and 7A-7D2, the stents include individual rings connected by connecting struts between layers. The design illustrated in the aforementioned drawings use four rings and two connecting struts, though the number of rings and connecting struts may be adjusted as needed for sizing and expansion. The rings are formed from a sinusoidally-shaped wire. The number of sinusoidal ‘periods’ corresponds to the amount of expansion in the stent. An increase of periods in the distal layers of the stent allows for additional stent expansion. The connecting struts between rings are minimal to allow the individual rings to expand to different shapes/diameters without being inhibited by the expansion of other rings.

### Example 1.1. Stent Delivery and Connection to Other Devices

Preferably, stents must be deliverable to a bifurcation core and expandable to a desired shape. A balloon system that can deliver and expand the stent to desired shapes can work together with the stent. To this end, Applicant's previously-disclosed work in custom balloon forming is contributing to the development of novel balloon designs for delivery and expansion of the stent. In particular, a prior U.S. Pat. No. 8,900,207 by Applicants uses a dual-guidewire balloon for delivery to a bifurcation area. The primary features of this balloon are 1) two guidewires that extend into the main branch and side branch, 2) a very short nose which abuts the carina; and 3) a non-cylindrical balloon. The software could be used to measure a patient's bifurcation anatomy and recommend a particular stent.

### Example 1.2. Stent Production and Testing

The stents shown in FIGS. 3A-3D and 4-6 were designed to fit a vessel of 16 mm diameter, increasing to a 21 mm diameter (circular). The stent has a stent pattern, with sinusoidally-shaped rings connected via connection struts. The overall sinusoidal shape was scaled up from similar sinusoidally-shaped stent designs. The circumference needed for the intended diameters was calculated, and used to calculate the number of “periods” in the sinusoid shape. For the stent “layers” of larger diameter, the number of periods were calculated, and the sinusoidal shape was adjusted to be slightly “pre-crimped” so that the stent could be fabricated as a rectangular sheet.

The current design uses two connecting struts between rings to allow for flexibility, and four rings. The number and location of connecting struts can be adjusted as needed for expansion. The number of stent rings may also be adjusted to accommodate different tapering lengths and overall stent lengths.

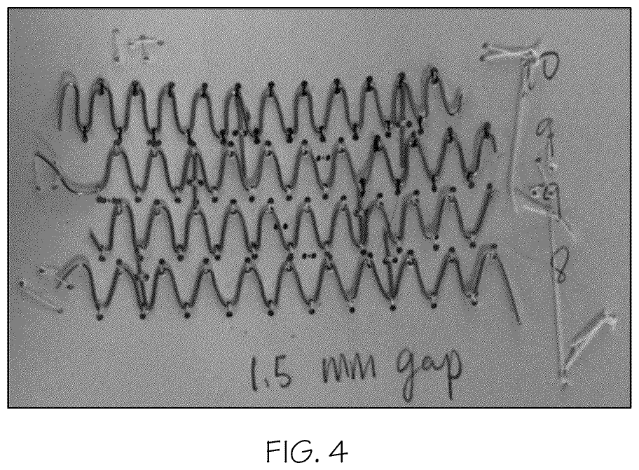

As illustrated in FIG. 4, the individual stent layers were formed by wrapping around nail boards. Cardstock stent layouts were laser-cut, and the individual stent layers were attached to the stent layout. Then the individual layers were laser-welded together into a sheet. Following the laser welding, the sheets were wrapped around a mandrel, trimmed, and the seam laser-welded to complete the stent.

Once the stent was complete, the stent was tested for expansion capability. Expansion tests involved crimping the stent onto a deflated balloon (FIG. 3A), and re-expanding the stent with a series of balloons (FIG. 3B). The stent was crimped and expanded to the intended shapes without any fracturing at welds or incomplete expansion. The stent recoiled (i.e., un-expand) at a rate of 10% or less for all expansions, with less ‘recoil’ as the diameter of expansion increased. The first expansions used a ‘conical’ balloon shape to confirm a circular cross section of 21 mm diameters was feasible.

Next, two tapered ‘kissing balloons’ were used to expand the stent to the 16 mm×26 mm ellipse shape (FIG. 3C). The success of these tests shows that the stent design allows for the intended variation in expansion diameter and an elliptical cross section with no observed issues.

The stents were tested for expandability and % elastic recoil (ER). The results are shown in Tables 1 and 2. The stents crimped 50% onto balloons. In test 1, the stents expanded using single tapered balloons. In test 2, the stents expanded using two tapered “kissing balloons.” The diameters were measured before and after balloon removal.

TABLE 1
Balloon dimensions for tests.

| | Proximal | Distal |
|---|---|---|
| | diameter | diameter |
| | (mm) | (mm) |
| Test 1 | 16 | 21 |
| Test 2 | 8 | 11 |
| Test 3 | 8 | 13 |

TABLE 2
Balloon dimensions for tests.

| | % Elastic | % Elastic |
|---|---|---|
| | Recoil | Recoil |
| | (Proximal) | (Distal) |
| Test 1 (16-21 mm) | 10 +/− 2% | 10 +/− 3% |
| Test 2 (8-11 mm KBI) | | |
| Long Axis | 4 +/− 0.6% | 11 +/− 4% |
| Short Axis | 4 +/− 7% | −4 +/− 4% |
| Test 3 (8-13 mm KBI) | | |
| Long Axis | 3 +/− 3% | 12 +/− 3% |
| Short Axis | 2 +/− 7% | 0 +/− 6% |

Additional designs may have distal/proximal ratios of 1.5 and 2, to accompany the 1.3 ratio. Additionally, positions of the connecting struts may vary.

For manufacturing, Applicants have bent and formed 316L stainless steel wires, which were laser-welded together. Other manufacturing methods, such as laser-cutting of hypo tubes, could be used. Alternatively, the wire bending/laser welding process could be automated. Other materials for the stent instead of stainless steel could be used. For example, thinner, stronger alloys such as cobalt and chromium could be utilized. Finally, drug coatings may be added to the stent.

In sum, this Example uses a novel stent design that is adaptable to the unique shapes of vessel (e.g., coronary) bifurcations. In particular, the stents are specifically designed for vessel bifurcations. As such, the stents can simplify the approach to bifurcation lesions and limit the amount of stent struts or overlapping stent struts. Together, this has the potential to improve patient outcomes and decrease procedural times.

### Example 2. Design and Validation of Additional Stents for Coronary Bifurcations

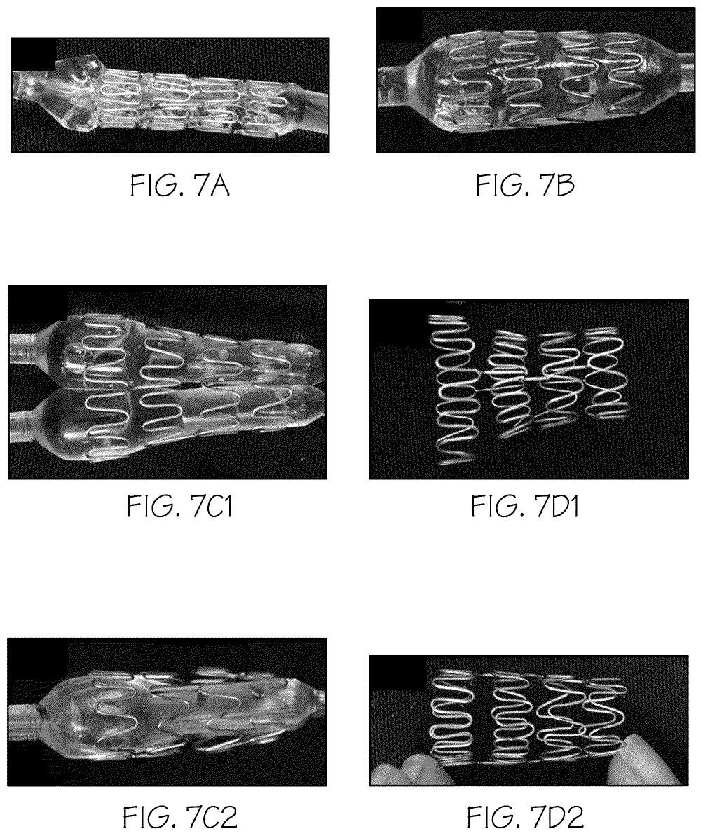

This Example provides expanded stents with balloons independently of a bifurcation model. FIGS. 7A-7D2 provide stent expansion tests. FIG. 7A shows a stent crimped onto a balloon used in Test 1. FIG. 7B shows a stent and balloon inflated during Test 1. FIGS. 7C1 and 7C2 show sample DKBI, including the long axis (FIG. 7C1) and the short axis of DKBI (FIG. 7C1). FIGS. 7D1 and 7D2 show stents following inflation tests, including the long axis of the stent (FIG. 7D1) and the short axis of the stent (FIG. 7D2). As summarized in Table 3, the elastic recoil (ER) of the stents on the proximal and distal ends of the stents were calculated.

TABLE 3
% ER of Stents.

| | Proximal | | Distal | | Total | |
|---|---|---|---|---|---|---|
| Long Axis | 7 ± 5 | (n = 7) | 13 ± 3 | (n = 7) | 10 ± 5 | (n = 14) |
| Short Axis | −1 ± 7 | (n = 7) | −3 ± 5 | (n = 7) | −2 ± 6 | (n = 14) |
| Total | 3 ± 7 | (n = 14) | 5 ± 9 | (n = 14) | 4 ± 8 | (n = 28) |

Mean ± Standard deviation of % ER.

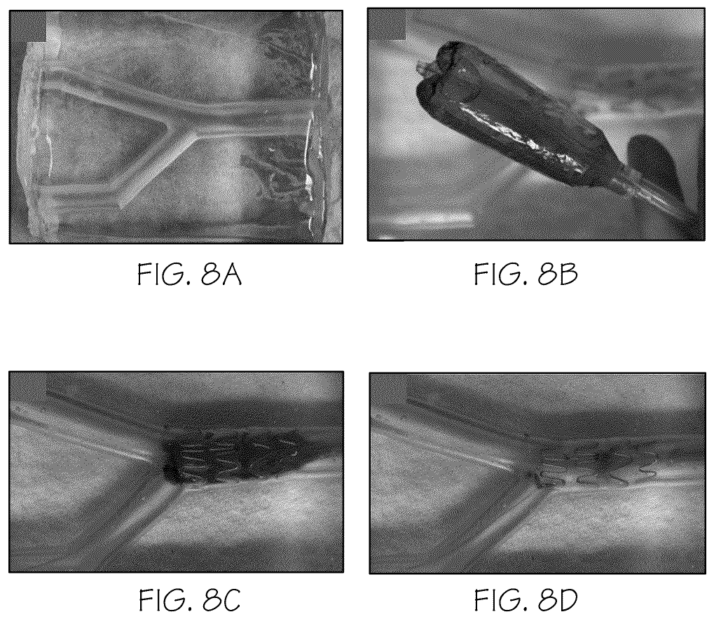

Further tests have placed the stents in a bifurcation model using balloon-catheter designs for stent delivery. This process is shown in FIGS. 8A-8D. FIG. 8A shows a phantom used in the test. FIG. 8B shows an exemplary balloon-catheter (Variation B). FIG. 8C shows a stent and balloon within the artery. FIG. 8D shows the placed stent in the artery while the balloon is retracted. As shown in FIG. 8C, the stents tend to be delivered closest to the carina.

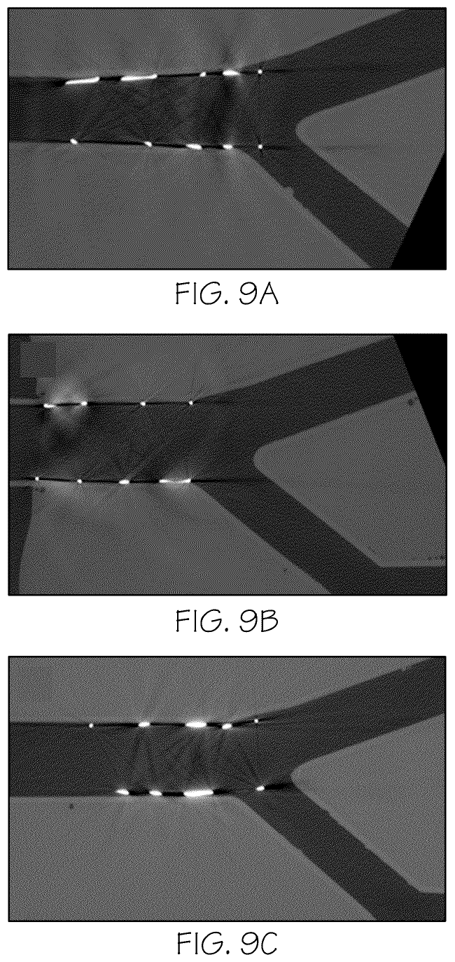

FIGS. 9A-9C show cross sections of bifurcation tests 1-3, respectively. The bright white spots indicate stent struts. Tests 1 and 3 saw stent edges nearest to the carina of the bifurcation. Following stent implantation near enough to the carina that the independent expansion of the stent rings is required, the stent needs to be expanded with bifurcation-specific balloon catheters.

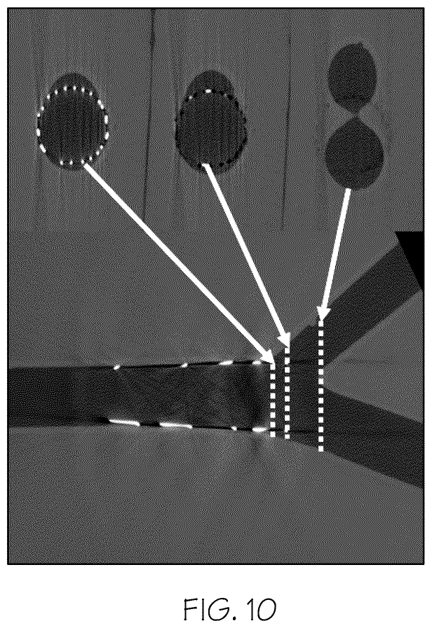

FIG. 10 shows the cross-sections, and the stent malapposition left near the carina of the bifurcation. In particular, FIG. 10 shows a MicroCT scan of Variation B. The cross sections of various locations in the pMV are shown, including the distal stent edge and the proximal carina edge. The bright white spots are the prototype stent struts, the light grey layer surrounding the vessel void is the 3D printed material and the darker gray surrounding the vessel is the gel.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein

1. A stent comprising:
a proximal end and a distal end; and
a plurality of interconnected and expandable rings positioned adjacent to one another from the proximal end to the distal end,
wherein the interconnected rings form a cavity,
wherein each ring is connected to and spaced apart from at least one adjacent ring through one or more connecting units, and
wherein the rings at or near the proximal end comprise different diameters than the rings at or near the distal end.

2. The stent of claim 1, wherein the stent is operable to receive at least one expanding structure in its cavity and be expanded by the at least one expanding structure, wherein the expanding structure comprises a balloon.

3. The stent of claim 2, wherein the expanding structure comprises a first balloon and a second balloon, wherein the first balloon is operable to extend into a main branch of a bifurcated vessel, and wherein the second balloon is operable to extend into a side branch of a bifurcated vessel.

4. The stent of claim 1, wherein the rings at or near the proximal end comprise larger diameters than the rings at or near the distal end.

5. The stent of claim 1, wherein the rings at or near the proximal end comprise smaller diameters than the rings at or near the distal end.

6. The stent of claim 1, wherein the rings comprise a sinusoidal shape, wherein the sinusoidal shape comprises alternating peaks and troughs along the ring length.

7. The stent of claim 1, wherein the connecting units comprise struts.

8. The stent of claim 1, wherein the stent comprises one to three connecting units between each adjacent ring.

9. The stent of claim 1, wherein the rings are connected to one another through connecting units to form a unitary structure without bifurcations.

10. The stent of claim 1, wherein the cavity comprises a non-uniform diameter from the proximal end to the distal end when the rings are expanded.

11. The stent of claim 10, wherein the cavity comprises a larger diameter at the proximal end than at the distal end when the rings are expanded.

12. The stent of claim 10, wherein the cavity comprises a larger diameter at the distal end than at the proximal end when the rings are expanded.

13. The stent of claim 12, wherein the diameter ratio of the cavity at the distal end relative to the cavity at the proximal end ranges from 1.2 to 2 when the rings are expanded.

14. A method of implanting a stent in a blood vessel of a subject, said method comprising:
positioning the stent at a desired location within the blood vessel; and
expanding the stent from a contracted configuration to an expanded configuration to secure the stent to the blood vessel wall, wherein the stent comprises:
a proximal end and a distal end, and
a plurality of interconnected and expandable rings positioned adjacent to one another from the proximal end to the distal end,
wherein the interconnected rings form a cavity,
wherein each ring is connected to and spaced apart from at least one adjacent ring through one or more connecting units, and
wherein the rings at or near the proximal end comprise different diameters than the rings at or near the distal end.

15. The method of claim 14, wherein the vessel is implanted at or near vessel bifurcations.

16. The method of claim 15, wherein the stent covers a proximal vessel of a bifurcated vessel up to a bifurcation carina.

17. The method of claim 15, wherein the stent covers a widening at a side-branch of a bifurcated vessel.

18. The method of claim 14, wherein the expanding of the stent comprises introducing at least one expanding structure into the cavity and expanding the expanding structure.

19. The method of claim 18, wherein the expanding structure comprises a first balloon and a second balloon, wherein the first balloon is operable to extend into a main branch of a bifurcated vessel, and wherein the second balloon is operable to extend into a side branch of a bifurcated vessel.

20. The method of claim 14, wherein the rings at or near the proximal end comprise larger diameters than the rings at or near the distal end.

21. The method of claim 14, wherein the rings at or near the proximal end comprise smaller diameters than the rings at or near the distal end.

22. The method of claim 14, wherein the method is utilized to treat or prevent a coronary artery disease
